(19) United States
(12) Patent Application Publication
(10) Pub. No.: US 2013/0253544 A1
(43) Pub. Date: Sep. 26, 2013

(54) SYSTEM, METHOD, AND APPARATUS FOR AN ANTERIOR PORTAL GUIDE FOR PARTIAL THICKNESS ROTATOR CUFF REPAIR

(71) Applicant: S. Charles Marshall, Jr., Sandy, UT (US)

(72) Inventor: S. Charles Marshall, Jr., Sandy, UT (US)

(21) Appl. No.: 13/831,511

(22) Filed: Mar. 14, 2013

Related U.S. Application Data
(63) Continuation of application No. 13/831,459, filed on Mar. 14, 2013.
(60) Provisional application No. 61/613,472, filed on Mar. 20, 2012.

(51) Int. Cl. A61B 17/04

(52) U.S. Cl. CPC A61B 17/0466
USPC 606/150

(57) ABSTRACT

An anchor placement jig for partial thickness rotator cuff repair. The anchor placement jig includes a frame, a target extending from a distal end of the frame, and a guide disposed on the frame and aligned with the target. The guide has a guide axis that intersects an aperture that extends through the target. The aperture is sized to receive a suture anchor. The target includes a perimeter structure. The perimeter structure extends at least partially around the aperture. The perimeter structure includes a perimeter clearance between the perimeter structure of the target and the aperture of less than one sixteenth of an inch.

### CROSS-REFERENCE TO RELATED APPLICATIONS

This application is a continuation of U.S. patent application Ser. No. 13/831,459, filed Mar. 14, 2013, which claims the benefit of U.S. Provisional Application No. 61/613,472, filed Mar. 20, 2012, which is hereby incorporated by reference herein in its entirety, including but not limited to those portions that specifically appear hereinafter, the incorporation by reference being made with the following exception: In the event that any portion of the above-referenced provisional application is inconsistent with this application, this application supersedes said above-referenced provisional application.

### STATEMENT REGARDING FEDERALLY SPONSORED RESEARCH OR DEVELOPMENT

Not Applicable.

### BACKGROUND

1. The Field of the Present Disclosure

The present disclosure relates generally to surgical tools, and more particularly, but not necessarily entirely, to a jig for rotator cuff repair.

2. Description of Related Art

The rotator cuff is a group of muscles and tendons that stabilize the shoulder joint. Injuries to the rotator cuff can result in pain and reduced movement of the arm. One common injury to the rotator cuff is a torn tendon. Torn tendons may include full thickness tears, in which the tendon is torn all the way through the tendon, and partial thickness chairs, in which the tendon is not completely torn.

A torn rotator cuff tendon is often repaired surgically. Historically, rotator cuff repairs were performed through an open incision. More recently, arthroscopic surgery has been used in rotator cuff repair. Arthroscopic surgery is much less invasive than traditional forms of surgery and can result in quicker recovery times and better outcomes.

In arthroscopic repair of a full thickness tear, a suture anchor may be inserted into the humerus for attaching the torn tendon. The tendon is then tied to this anchor using sutures, after which it becomes secured to the surrounding bone. Proper placement of the anchor is critical to a successful outcome.

In arthroscopic repair of a partial thickness tear, determination of a desirable anchor placement location is restricted by the still connected portion of the tendon. In order to properly place the suture anchor to repair the partial thickness tear, surgeons often complete the partial thickness tear to create a full thickness tear. After completing the tear, the surgeon can more easily determine the desired anchor placement location. While completing the tear of a partial thickness tear allows the surgeon to correctly place the suture anchor in a desired location on the humerus, the additional tear results in longer recovery times and less desirable outcomes.

Some surgeons have developed skills for placing suture anchors in desirable locations in partial thickness to rotator cuff tear repairs. Blind placement accuracy requires a significant amount of practice and can result in less desirable outcomes while the surgeon is developing this skill. Consequently, many surgeons do not attempt blind placement, instead relying on completing partial thickness tears to more easily access the desired anchor location.

Since the difficulty associated with blind placement of suture anchors has resulted in many partial thickness tears being converted to full thickness tears, many patients are subjected to a longer recovery times and potentially less desirable results. Consequently, a way for improving blind placement accuracy without requiring additional difficult skill development is desirable.

The features and advantages of the present disclosure will be set forth in the description which follows, and in part will be apparent from the description, or may be learned by the practice of the present disclosure without undue experimentation. The features and advantages of the present disclosure may be realized and obtained by means of the instruments and combinations particularly pointed out in the appended claims.

### BRIEF DESCRIPTION OF THE DRAWINGS

The features and advantages of the disclosure will become apparent from a consideration of the subsequent detailed description presented in connection with the accompanying drawings in which:

FIG. 1 is a perspective view of one embodiment of a an anchor placement jig made in accordance with the principles of the present disclosure;

FIG. 2 is a side view of the anchor placement jig of FIG. 1;

FIG. 3 is a side view of an alternative embodiment of an anchor placement jig;

FIG. 4 is a top view of one embodiment of a target of the anchor placement jig of FIG. 1;

FIG. 5 is a side view of one embodiment of the target of the anchor placement jig of FIG. 1;

FIG. 6 is a front view of the anchor placement jig of FIG. 1;

FIG. 7 is a front view of an alternative embodiment of an anchor placement jig;

FIG. 8 is an posterior view of bone and muscle tissue in a patient's shoulder with one embodiment of an anchor placement jig;

FIG. 9A is an anterior view of a partial thickness tear of a rotator cuff tendon with a positioned target of one embodiment of an anchor placement jig;

FIG. 9B is an anterior view of a partial thickness tear of a rotator cuff tendon with a suture anchor installed in the humerus;

FIG. 9C is an anterior view of a repaired partial thickness tear of a rotator cuff tendon;

FIG. 10 is a flowchart diagram showing one embodiment of a method for repairing a partial thickness rotator cuff tear using an anchor placement jig;

FIG. 11 is a side view of one embodiment of an anchor placement jig;

FIG. 12 is a side view of an instrument for use with the anchor placement jig depicted in FIG. 11;

FIG. 13 is a cross-sectional view of the instrument installed into a cannulated target arm of the anchor placement jig depicted in FIG. 11; and

FIG. 14 is an end view of the cannulated target arm of the anchor placement jig depicted in FIG 11.

### DETAILED DESCRIPTION

For the purposes of promoting an understanding of the principles in accordance with the disclosure, reference will now be made to the embodiments illustrated in the drawings and specific language will be used to describe the same. It will nevertheless be understood that no limitation of the scope of the disclosure is thereby intended. Any alterations and further modifications of the inventive features illustrated herein, and any additional applications of the principles of the disclosure as illustrated herein, which would normally occur to one skilled in the relevant art and having possession of this disclosure, are to be considered within the scope of the disclosure claimed.

Before the present systems, methods, and apparatuses for an anchor placement jig are disclosed and described, it is to be understood that this disclosure is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present disclosure will be limited only by the appended claims and equivalents thereof.

It must be noted that, as used in this specification and the appended claims, the singular forms “a,” “an,” and “the” include plural referents unless the context clearly dictates otherwise.

In describing and claiming the present disclosure, the following terminology will be used in accordance with the definitions set out below.

As used herein, the terms “comprising,” “including,” “containing,” “characterized by,” and grammatical equivalents thereof are inclusive or open-ended terms that do not exclude additional, unrecited elements or method steps.

As used herein, the term “proximal” shall refer broadly to the concept of a nearest portion. For example, the grip is the proximal-most portion of the anchor placement jig because it is the nearest portion when the anchor placement jig is in use.

As used herein, the term “distal” shall generally refer to the opposite of proximal, and thus to the concept of a further portion, or a furthest portion, depending upon the context.

Applicant has discovered that proper placement of a suture anchor in an arthroscopic repair of a partial thickness rotator cuff tear can be more easily and consistently achieved through the use of an anchor placement jig. Applicant has thus conceived of an anchor placement jig that includes a target that can be positioned at a desired location on the humerus. The jig may also include a guide to direct insertion and installation of a suture anchor. The guide is aligned with the target, such that a suture anchor installed using the guide is installed at the target.

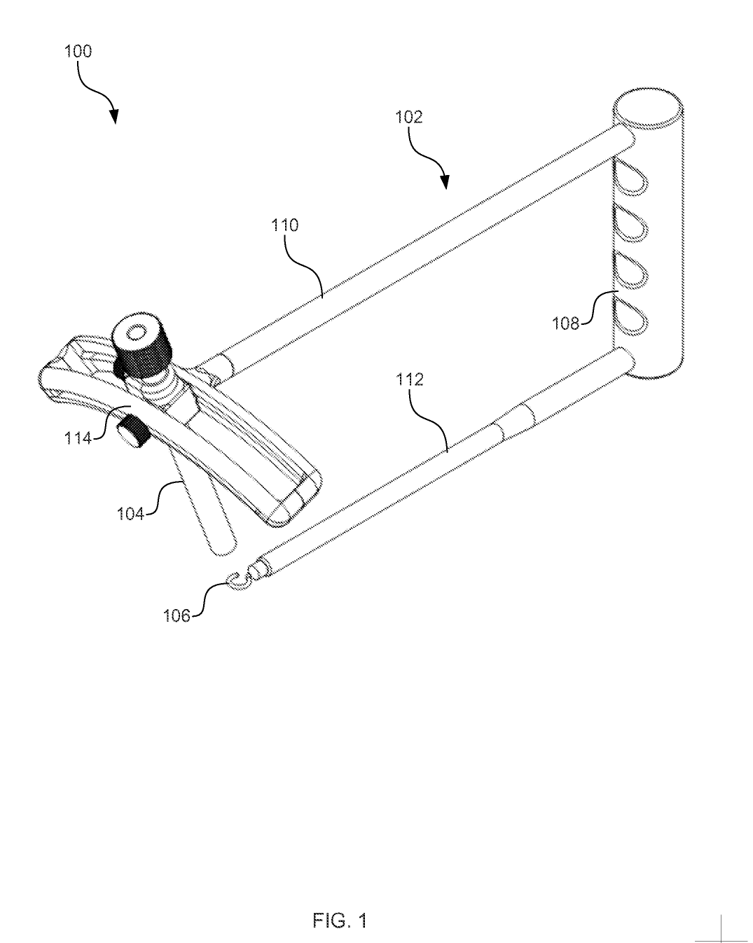

Referring now to the drawings, FIG. 1 is a perspective view of one embodiment of an anchor placement jig 100 made in accordance with the principles of the present disclosure. The anchor placement jig 100 includes a frame 102, a guide 104, and a target 106. The anchor placement jig 100 facilitates insertion of a suture anchor at a position near the target 106.

The frame 102, in some embodiments, includes a vertical grip 108, a guide arm 110, and a target arm 112. The frame 102 holds the guide 104 in a position aligned with the target 106 such that a suture anchor installed using the guide 104 is positioned at or near the target 106.

In some embodiments, the frame 102 includes a vertical grip 108. The vertical grip 108 provides a structure that a surgeon may use to comfortably hold the frame 102 while using the anchor placement jig 100. The vertical grip 108 may be essentially perpendicular to one or more other elements of the frame 102. In some embodiments, the vertical grip 108 is contoured to make holding and manipulating the anchor placement jig 100 more comfortable.

The frame 102, in some embodiments, includes a guide arm 110. The guide arm 110 extends to a distal end of the frame 102 and provides a connection point for the guide 104. The guide arm 110 may be substantially perpendicular to the vertical grip 108. In some embodiments, the guide arm 110 is substantially straight.

In certain embodiments, the frame 102 includes a target arm 112. The target arm 112 extends to a distal end of the frame 102 and provides a connection point for the target 106. The target arm 112 may be substantially perpendicular to the vertical grip 108. In some embodiments, the target arm 112 is substantially straight. The target arm 112 may be thinner at a distal end of the target arm 112 than it is at a proximal end of the target arm 112. The target arm 112 may include a step where the thickness of the target arm 112 changes. The target arm 112 may include a tapered region where the thickness of the target arm 112 changes. The target arm 112 may include a taper extending substantially the entire length of the target arm 112.

The frame 102 may include any material strong and rigid enough the hold the guide 104 in a position aligned with the target 106. For example, the frame 102 may include stainless steel, such as a precipitation hardened stainless steel. Other examples of materials suitable for use in the fame 102 include titanium, composite materials such as carbon fiber in a polymer matrix, or a rigid polymer material.

The guide 104, in one embodiment, guides a suture anchor to a position near or within the target 106. In one embodiment, the guide 104 is a barrel or tube, having a cylindrical wall and a hollow center with openings at the top and the bottom. The guide 106 is disposed at a distal end of the frame 102. The guide 106 is disposed at the distal end of the guide arm 110, in some embodiments.

The target 106, in one embodiment, is positionable at a desired location for installing a suture anchor. While the target 106 is positioned at the desired location, a suture anchor may be guided to the target 106 via the guide 104. The target 106 is disposed at a distal end of the frame 102. The target 106 extends from the distal end of the target arm 112, in some embodiments such that the target 106 may or may not be fixedly connected to the distal end of the target arm 112.

In some embodiments, the anchor placement jig 100 includes a guide rotation adjustment mechanism 114. The guide rotation adjustment mechanism 114 is releasable such that the guide 104 may be rotated about the target 106. As the guide 104 is rotated, it remains aligned with the target 106. The guide rotation adjustment mechanism 104 is securable such that the guide 104 does not rotate, but remains in a secured position. By rotating the guide 104 to a desired position via the guide rotation adjustment mechanism 104, the surgeon may adjust the position in which the anchor placement jig 100 is held while in use. The adjustable position may improve comfort for the surgeon or allow better control of placement of the target 106.

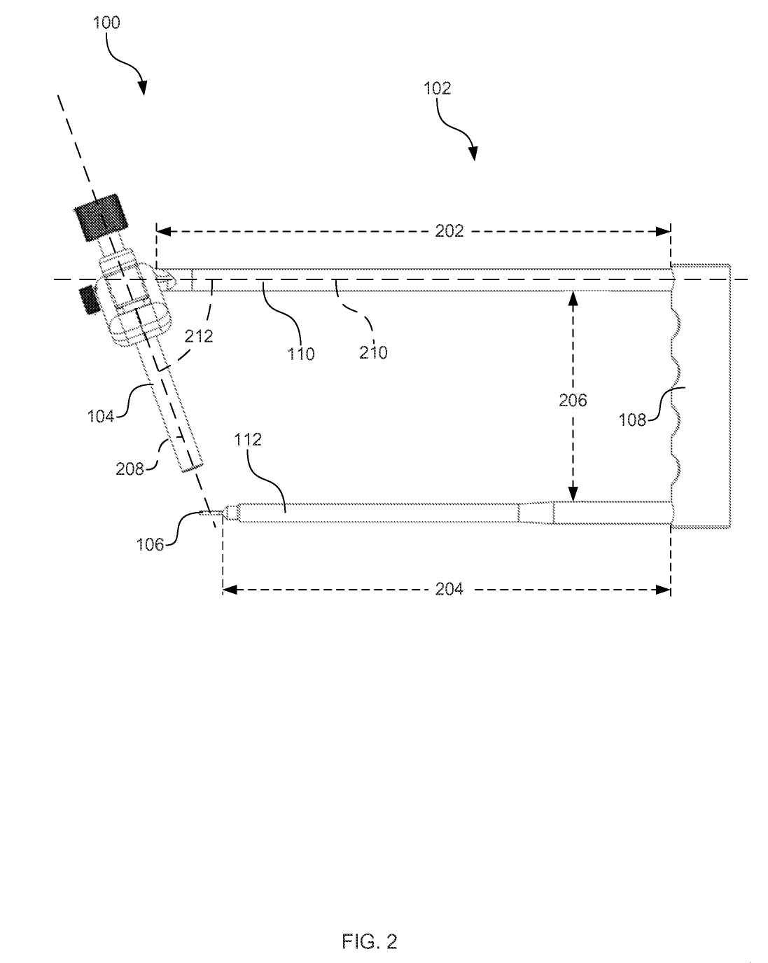

FIG. 2 is a side view of the anchor placement jig 100 of FIG. 1. The anchor placement jig 100 includes a guide 104, a target 106, a vertical grip 108, a guide arm 110, and a target arm 112. The guide 104, target 106, vertical grip 108, guide arm 110, and target arm 112 are similar to same numbered components described in relation to FIG. 1.

In certain embodiments, the guide arm 110 has a guide arm length 202 defined from the connection point of the guide arm 110 with the guide 104 to the connection point of the guide arm 110 to other components of the anchor placement jig 100. In certain embodiments, the guide arm length 202 is between eight and ten inches. In one embodiment, the guide arm length 202 is approximately nine inches.

The target arm 112 has a target arm length 204 defined from the connection point of the target arm 112 with the target 106 to the connection point of the target arm 204 to other components of the anchor placement jig 100. In certain embodiments, the target arm length 204 is between seven and nine inches. In one embodiment, the target arm length 204 is approximately eight inches.

In some embodiments, the guide arm 110 and the target arm 112 are substantially parallel along at least a portion of the guide arm 110 and at least a portion of the target arm 112. In one embodiment, the guide arm 110 and the target arm 112 are substantially parallel along substantially their entire lengths.

The guide arm 110 and the target arm 112 may be separated by a clearance distance 206 along at least a portion of their respective lengths. The clearance distance 206 allows for clearance of the anchor placement jig 100 around the tissues covering the humerus during arthroscopic surgery. In one embodiment, the target arm 112 may be separated by a clearance distance 206 along substantially their entire lengths. The clearance distance 206, in some embodiments, is between two and four inches. In one embodiment, the clearance distance 206 is approximately three inches.

The guide 104, in some embodiments, defines a guide axis 208 that runs substantially along the center of the guide 104 along the longest dimension of the guide 104. The guide axis 208 may run substantially along the path that the suture anchor is installed following the guide 104. In some embodiments, the guide axis 208 is substantially straight. The guide arm 110 may define a long axis of the guide arm 210. The long axis of the guide arm 210 may run substantially along the longest dimension of the guide arm 110.

In some embodiments, the guide axis 208 and the long axis of the guide arm 210 form a guide angle 212. The guide angle 212, in some embodiments, is substantially ninety degrees. In an alternative embodiment, the guide angle 212 is less than ninety degrees. In one embodiment, the guide angle 212 is approximately seventy degrees.

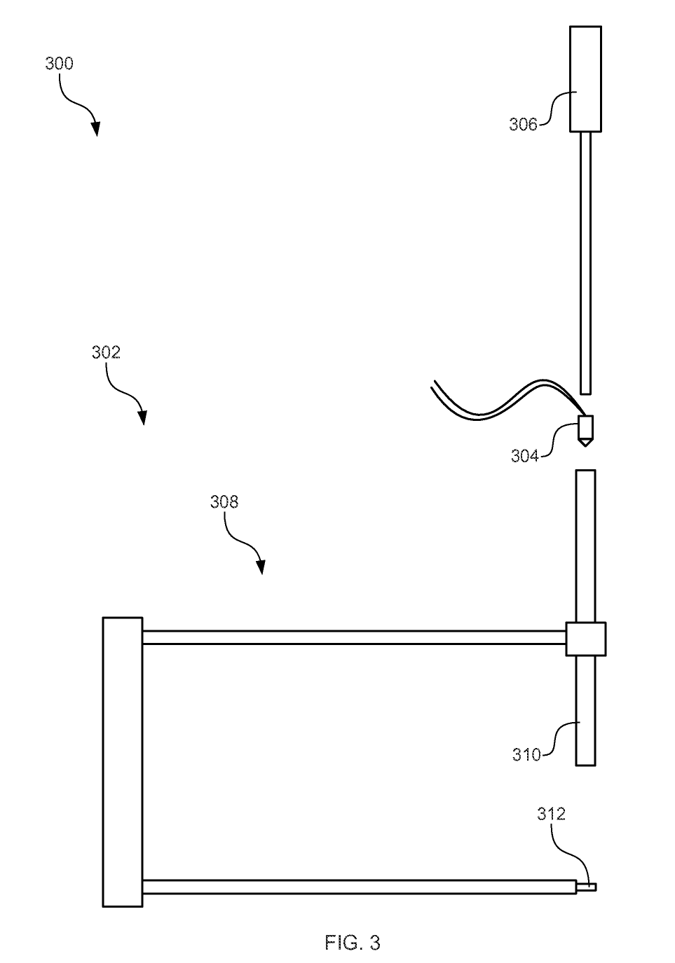

FIG. 3 is a side view of a system 300 for repairing a partial thickness rotator cuff tear. The system 300 includes an anchor placement jig 302, a suture anchor 304, and a driver 306. The system 300 facilitates placement of a suture anchor 304 to repair a partial thickness rotator cuff tear.

The anchor placement jig 302, in one embodiment, includes a frame 308, a guide 310, and a target 312. The guide 310 and the target 312 are connected to and held in place by the frame 308. The guide 310 is aligned with the target 312. In one embodiment, the guide 310 does not rotate with respect to the frame 308. In some embodiments, the guide 310 is adjustable in an “up” and “down” direction, meaning that the distance between the guide 310 and the target 312 may be adjusted.

The suture anchor 304, in one embodiment, is a bone anchor with an attachment point for one or more sutures. The suture anchor 304 may be any type of suture anchor. For example, the suture anchor 304 may be a metal anchor with a screw mechanism, in which the suture anchor 304 is rotated to fix the suture anchor 304 in the humerus. In some embodiments, the suture anchor 304 includes biodegradable material that dissolves in the body over time. In one embodiment, the suture anchor 304 is an interference fit suture anchor that is secured by pressing into the humerus.

The suture anchor 304 may include a suture connection point for attaching one or more sutures. The suture connection point may be any type of connection point. For example, the suture connection point may be an eyelet.

The suture anchor 304 may include any number or type of sutures. For example, the suture anchor 304 may include four sutures made of nylon. In one embodiment, the suture anchor 304 includes two sutures with each suture having two ends threaded through an eyelet.

In some embodiments, the driver 306 is a tool for installing the suture anchor 304. The driver 306 may be a cannulated driver such that sutures connected to the suture anchor 304 may be threaded through the driver 306 during installation. The driver 306 may interface with the suture anchor 304 to rotate the suture anchor 304 for installation. In an alternative embodiment, the driver 306 interfaces with the suture anchor 304 to press the suture anchor 304 for installation.

In some embodiments, the suture anchor 304 is installable through the guide 310 using the driver 306. The guide 310 directs the suture anchor 304 to the target 312. By placing the target 312 at a desired location on the humerus, then guiding the suture anchor 304 to the target 312 using the guide 310, surgeons can accurately place the suture anchor 304 in a desired location and orientation.

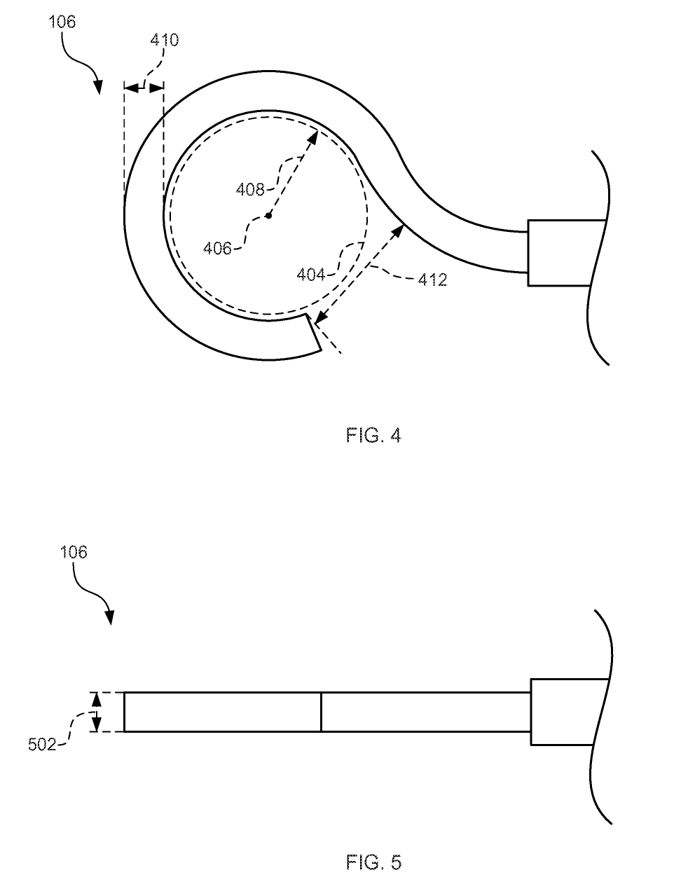

FIG. 4 is a top view of one embodiment of a target 106 of the anchor placement jig 100 of FIG. 1. The target 106 includes a perimeter structure 402. The target 106 is a structure that indicates a location where a suture anchor 304 installed via the guide 104 will be placed.

The target 106, in some embodiments, is formed into a hook configuration. The perimeter structure 402 may be curved such that the perimeter structure 402 forms a hook. The perimeter structure 402 may at least partially surround a space at the center of the perimeter structure 402 to form an aperture 404.

The aperture 404 may be sized to receive a suture anchor 304. The aperture 404 may be substantially the same size as a cross-section of the suture anchor 304 or larger than a cross section of the suture anchor 304. In some embodiments, an interior border of the perimeter structure 402 defines the aperture 404. In some embodiments, the aperture 404 is substantially circular, having a center 406 and a diameter 408. In one embodiment, the diameter 408 is between one eighth of an inch (⅛″) and one half of an inch (½″). The aperture 404, in one embodiment, has a maximum diameter of one quarter inch (¼″).

In an alternative embodiment, the aperture 404 is non-circular. For example, the aperture 404 may be substantially elliptical. In another example, the aperture 404 may be polygonal.

The perimeter structure 402 in some embodiments, has a perimeter clearance 410. The perimeter clearance 410 may be defined by a distance between the interior border of the perimeter structure 402 and the exterior border of the perimeter structure 402. In one embodiment, the perimeter clearance 410 is substantially constant throughout the entire perimeter structure 402. In an alternate embodiment, the perimeter clearance 410 varies along the perimeter structure 402. In one embodiment, the perimeter clearance 410 is between one sixty fourth of an inch ( 1/64″) and one sixteenth of an inch ( 1/16″) in at least one location along the perimeter structure 402. In one embodiment, the perimeter clearance 410 is approximately one thirty second of an inch ( 1/32″). The perimeter clearance 410 may be relatively small in order to place the aperture 404 in a location relatively close to tissues in the shoulder, such as a connected portion of a partially-torn tendon.

In certain embodiments, the perimeter structure 402 does not entirely surround the aperture 404. The portion of the aperture 404 that is not surrounded by the perimeter structure 402 defines a gap 412. In one embodiment, the gap 412 is sized such that it forms an opening through which a suture constrained at both ends may be passed. The constrained suture may be passed into the aperture 404 or out of the aperture 404 via the gap.

In one embodiment, the target 106 is connected to the frame 102 such that the target 106 is constrained relative to the frame 102. In another embodiment, the target 106 is rotatable around an axis running the length of the target arm 112. In a further embodiment, the target 106 is removable from the frame 102. In some embodiments, the target 106 may be selected from a plurality of different-sized targets to match a desired size of suture anchor, and the selected target 106 may be removably attached to the frame 102.

FIG. 5 is a side view of one embodiment of the target 106 of the anchor placement jig 100 of FIG. 1. The target 106 includes a perimeter structure 402. The target 106 is a structure that indicates a location where a suture anchor 304 installed via the guide 104 will be placed.

In some embodiments, the perimeter structure 402 has a height 502. The height 502 is defined by a distance between a superior border and an inferior border of the perimeter structure 402. In one embodiment, the height 502 is substantially constant along the perimeter structure 402. In another embodiment, the height 502 varies along the perimeter structure 402. The height 502, in some embodiments, is between one sixty fourth of an inch ( 1/64″) and one sixteenth of an inch ( 1/16″). In some embodiments, the height 502 is approximately one thirty second of an inch ( 1/32″). In certain embodiments, the height 502 is relatively small in order to place the aperture 404 in a location relatively close to tissues in the shoulder, such as a connected portion of a partially-torn tendon.

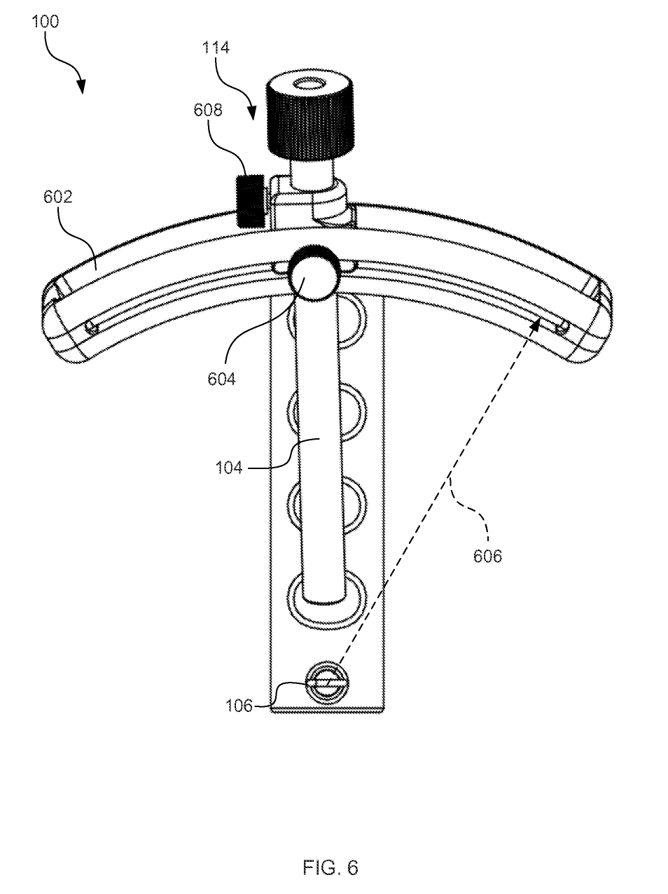

FIG. 6 is a front view of the anchor placement jig 100 of FIG. 1. The anchor placement jig 100 includes a guide rotation adjustment mechanism 114 including a rotation track 602 and a rotation adjustment release 604. The guide rotation adjustment mechanism 114 allows the guide 104 to be rotated around the target 106 while maintaining the guide 104 in alignment with the target 106. The guide rotation adjustment mechanism 114 allows the surgeon to place the anchor placement jig 100 in a position to improve surgeon comfort while placing the target 106 in a desired location.

The rotation track 602 provides a pathway for the guide 104 to travel while rotating around the target 106. The rotation track 602 may include one or more curved slots to accept one or more elements of the guide 104. The rotation track 602 may orient the guide 104 such that it is aligned with the target 106 in all positions along the rotation track 602. In one embodiment, the rotation track 602 includes one or more elements that follow a constant rotation radius 606 from the target 106.

The rotation adjustment release 604, in one embodiment, is a release that allows the guide 104 to travel along the rotation track 602 in response to the rotation adjustment release 604 being released. The guide 104 may be fixed to one location on the rotation track 602 in response to the rotation adjustment release 604 being engaged. For example, the rotation adjustment release 604 may be a screw that engages a surface of the rotation track 602 when engaged. The screw may further not engage the rotation track 602 when the screw is unscrewed, thus freeing the guide 104 to travel along the rotation track 602.

In some embodiments, the guide 104 is further adjustable via an insertion adjustment release 608. The insertion adjustment release 608 allows the guide 104 to be adjusted closer to or further from the target 106 in response to the insertion adjustment release 608 being released. The guide 104 may be fixed to a particular distance from the target 106 in response to the insertion adjustment release 608 being engaged. For example, the insertion adjustment release 608 may be a screw that engages a surface of the guide 104 when engaged. The screw may further not engage the guide 104 when the screw is unscrewed, thus freeing the guide 104 to travel closer to or further from the target 106.

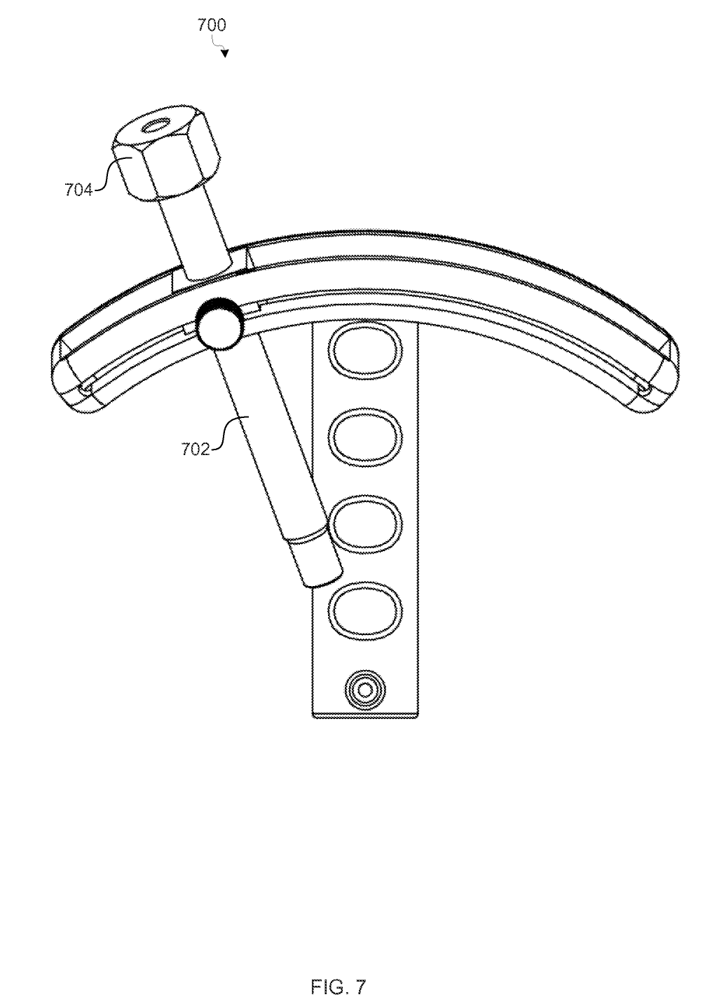

FIG. 7 is a front view of an alternative embodiment of an anchor placement jig 700. The anchor placement jig 700 includes a guide 702 having a hexagonal engagement structure 704. The hexagonal engagement structure 704 is engageable via a tool, such as a wrench. In an alternative embodiment, the guide 702 may have a round engagement structure. In some embodiments, the engagement structure is textured.

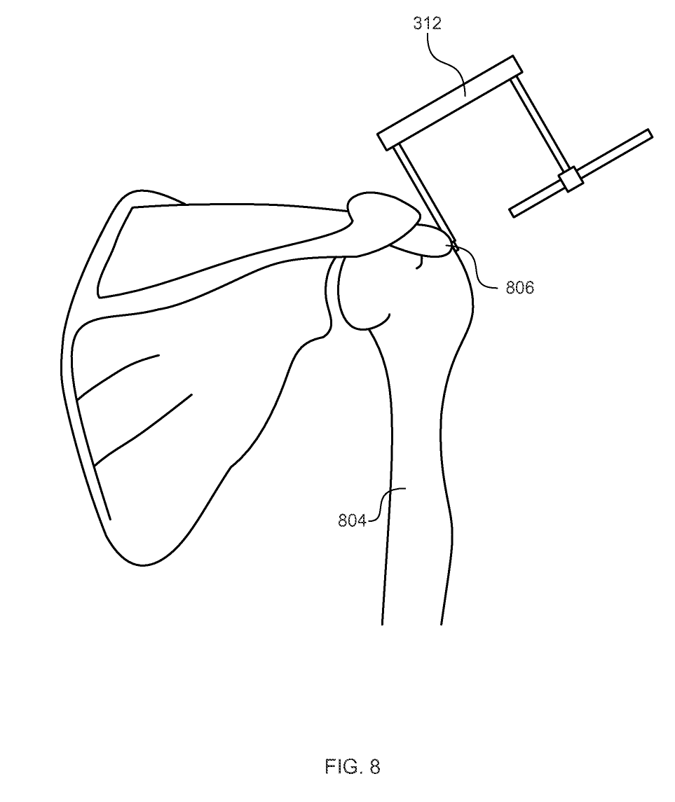

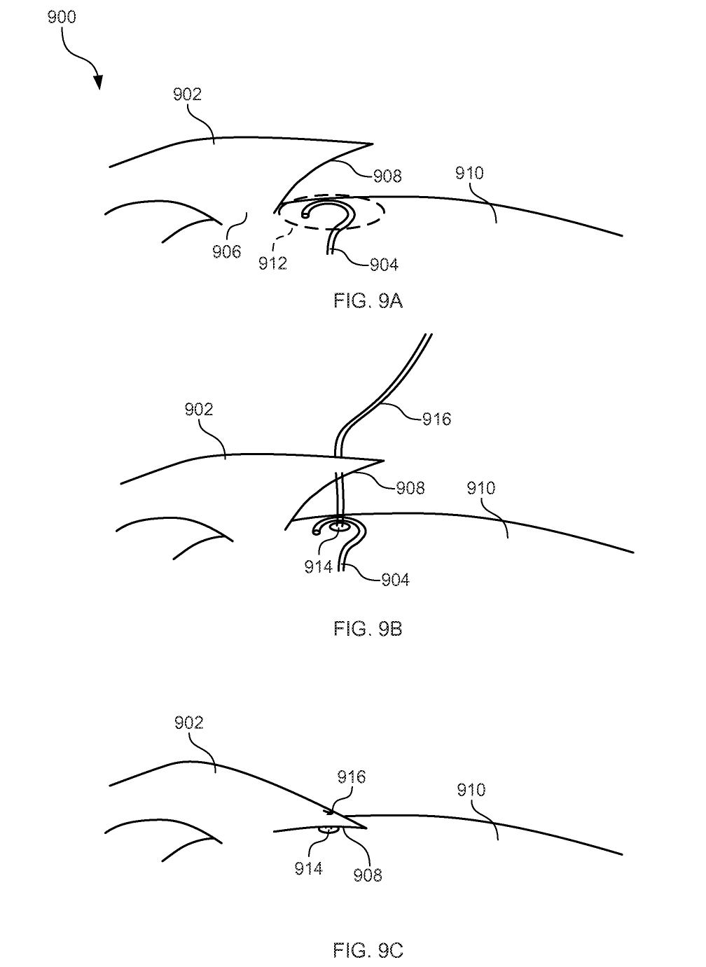

FIG. 8 is an posterior view of bone and muscle tissue in a patient's shoulder with one embodiment of an anchor placement jig 802. The anchor placement jig 802 is partially inserted through an anterior portal such that the target of the anchor placement jig 802 is positioned in a desired location on the humerus 804. The desired location may be a location where a tendon 806 will be attached to the humerus 804.

FIG. 9A is an anterior view of a partial thickness tear 900 of a rotator cuff tendon 902 with a positioned target 904 of one embodiment of an anchor placement jig. The partial thickness tear 900 includes a connected portion 906 of the tendon 902 and a torn portion 908 of the tendon 902. The connected portion 906 remains connected to the humerus 910, while the torn portion 908 is no longer connected to the humerus 910.

The target 904 may be positioned on the humerus 906 in a desired location 912 for attaching the torn portion 908 of the tendon 902 to the humerus 910. The target 904 is positioned at the desired location 912 by passing the target 904 through a portal. In one embodiment, the target 904 is passed through an anterior portal.

FIG. 9B is an anterior view of a partial thickness tear 900 of a rotator cuff tendon 902 with a suture anchor 914 installed in the humerus 910. In some embodiments, the suture anchor 914 is installed in the humerus 910 by guiding it to the target 904 using a guide of a suture anchor placement jig. The suture anchor 914 may have one or more attached sutures 916. The sutures 916 may be retrievable through the portal by withdrawing the target 904. In one embodiment, the sutures 916 are drawn through the torn portion 908 of the tendon 902.

FIG. 9C is an anterior view of a repaired partial thickness tear 900 of a rotator cuff tendon 902. The sutures 916 may be tied such that the torn portion 908 of the tendon 902 is drawn into and held in contact with the humerus 910. The torn portion 908 may then reattach to the humerus 910, repairing the partial tear.

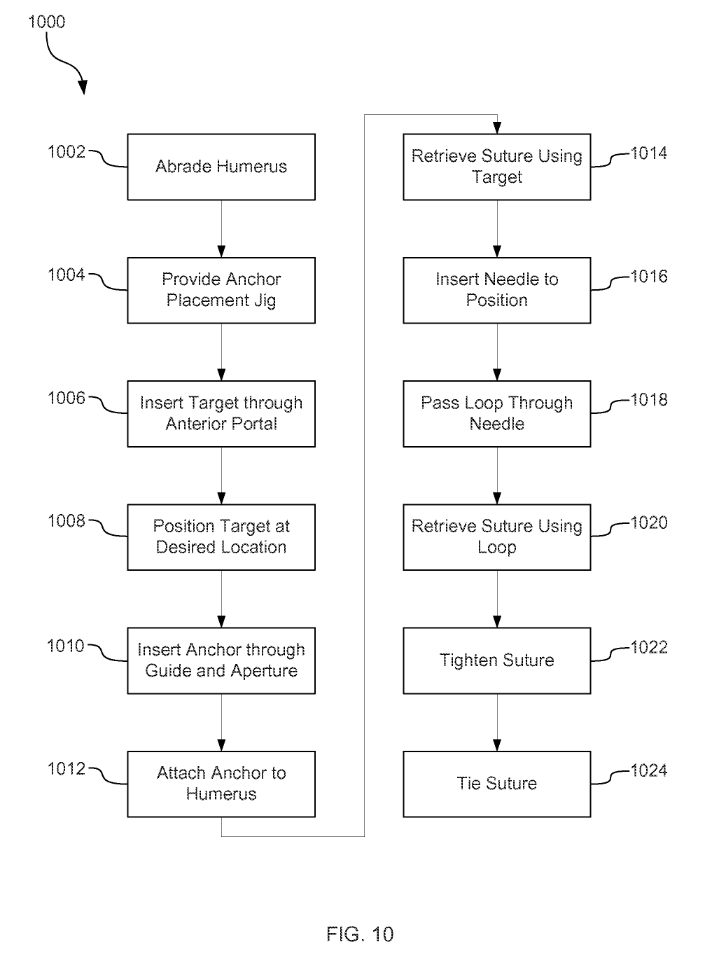

FIG. 10 is a flowchart diagram showing one embodiment of a method 1000 for repairing a partial thickness rotator cuff tear using an anchor placement jig. The method 1000 is in certain embodiments a method of use of the system and apparatus of FIGS. 1-9, and will be discussed with reference to those figures. Nevertheless, the method 1000 may also be conducted independently thereof and is not intended to be limited specifically to the specific embodiments discussed above with respect to those figures.

In some embodiments, an abrasion region on the humerus is abraded 1002. Abrading 1002 the abrasion region may remove extraneous material and stimulate the abrasion region to encourage attachment of the torn portion 910 of the tendon 902. In one embodiment, the abrasion region is located at the desired location 912 for placing the suture anchor 914. In some embodiments, the abrasion region includes the desired location 912 for placing the suture anchor 914.

An anchor placement jig is provided 1004 for placing the suture anchor 914 in the desired location 912. The anchor placement jig may include a target 904 and a guide aligned with the target 914.

The target 904 is inserted 1006 through a portal in the patient. In some embodiments, the target 904 is inserted 1006 through an anterior portal. The target is positioned 1008 at the desired location 912 of the humerus 910. In some embodiments, the position of the target 904 is viewed through an arthroscopic surgery camera. The surgeon may position 1008 the target 904 while viewing the output of the arthroscopic surgery camera.

The suture anchor 914 is inserted 1010 through the guide of the anchor placement jig and further through an aperture of the target 904. The guide directs the suture anchor 914 to the target 904 and through the aperture of the target 904 to the desired location 912 on the humerus 910.

The suture anchor 914, positioned by the anchor placement jig, is attached 1012 to the humerus 910 in the desired location 912. The suture anchor 914 may be attached 1012 using any method, including, but not limited to rotating the suture anchor 914 and pressing the suture anchor 914.

In some embodiments, one or more sutures 916 connected to the suture anchor 914 are retrieved 1014 using the target 904. The sutures 916 may be retrieved 1014 by withdrawing the target 904 from the portal.

A needle may be inserted 1016 through the torn portion 908 of the tendon 902 and positioned near the suture anchor 914. The position of the needle relative to the tendon 902 and the suture anchor 914 may be viewed through an arthroscopic surgery camera. In some embodiments, the needle is an eighteen gauge needle. A loop may be passed 1014 through the needle to a position near the suture anchor 914. The loop may be used to retrieve 1020 one or more sutures 916 connected to the suture anchor 914. In some embodiments, steps 1016 through 1020 may be repeated one or more times to thread sutures 916 through the torn portion 908 of the tendon 902 in a plurality of locations.

The sutures 916 may be tightened 1022 on the torn portion 908 of the tendon 902 to draw the torn portion 908 into contact with the abrasion region. The sutures 916 may then be tied 1024 to secure the torn portion 908 on the abrasion region.

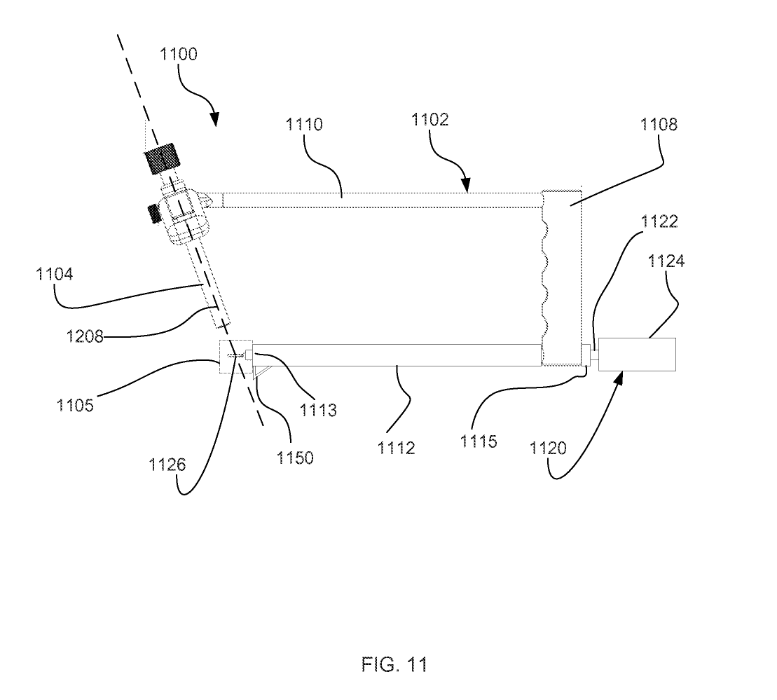

Referring now to FIG. 11, a side view of an anchor placement jig 1100 made in accordance with the principles of the present disclosure is shown. The anchor placement jig 1100 includes a frame 1102. The frame 1102 may include a guide arm 1110 and a target arm 1112. The guide arm 1110 and the target arm 1112 may be separated by a distance similar to that of the anchor placement jig 100 described above. The guide arm 1110 and the target arm 1112 may extend from a vertical grip 1108. The vertical grip 1108 may take the same form, and have the same function as, the vertical grip 108 described above. In addition, the target arm 1112 may extend through the vertical grip 1108.

Disposed on the free end of the guide arm 1110 may be a guide 1104. The guide 1104 may be in a position aligned with an anchor target area 1105 such that a suture anchor installed using the guide 1104 is correctly positioned at or near the anchor target area 1105. The guide 1104 may be positionable in the same manner as the guide 104 described above. The guide 1104 may include a guide barrel. In an embodiment, the target arm 1112 may be cannulated to allow passage of an instrument 1120.

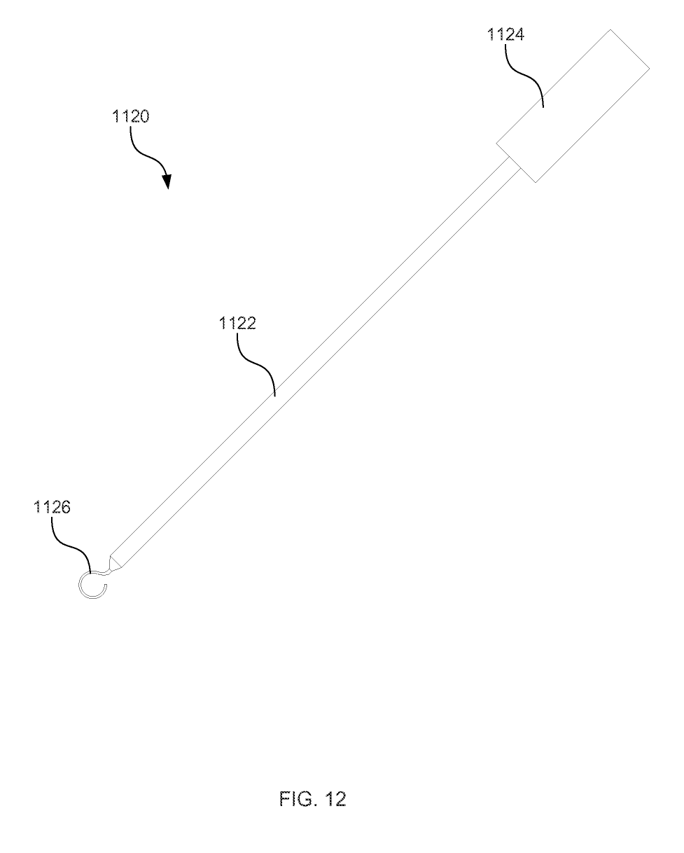

Referring now to FIGS. 11 and 12, there is depicted an instrument 1120 pursuant to an embodiment of the present disclosure. The instrument 1120 may include an elongated portion 1122. In an embodiment, the elongated portion 1122 may include a rigid rod or a flexible member, or any suitable elongate member. Disposed on a proximal end of the elongated portion 1122 may be a grip 1124. It will be appreciated that the grip 1124 allows a surgeon to manipulate the position of the instrument 1120 in the cannulated target arm 1112.

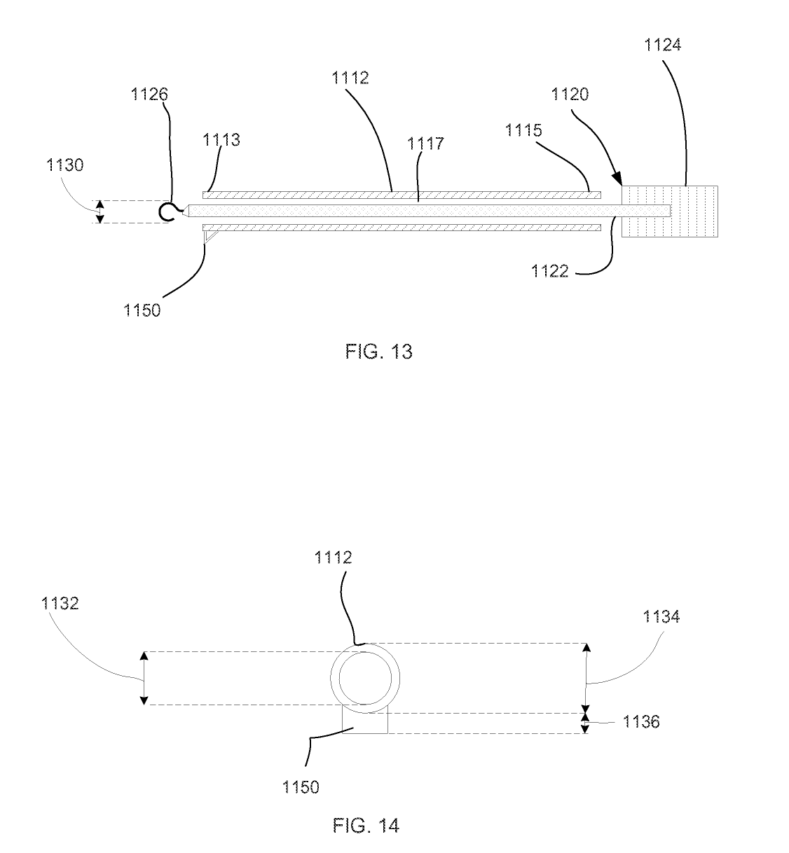

In an embodiment, it is to be understood that the instrument 1120 is part of the frame 1102, and thus constitutes a moveable portion of the frame 1102. In an embodiment, the instrument 1120 is therefore part of the frame 1102 when it resides within the cannulated target arm 1112 as shown in FIG. 13, and when it is free of and does not reside within the cannulated target arm 1112, as shown in FIG. 12.

Extending from the end of the instrument 1120 may be a tool head 1126. In an embodiment, the tool head 1126 may be a target, similar in form and function to the target 106 described above. It will be appreciated that the target may be any structure that indicates the target location for the guide 1104. In an embodiment, the tool head 1126 may take the form of a hook. In an embodiment, the tool head 1126 may take the form of a suture grabber. In an embodiment, the tool head 1126 may take the form of tool heads suitable for use in minimally invasive surgery, such as micro-tools, suture grabbers, bayonets, scissors, blades, forceps, distractors, spreaders, and clamps. In an embodiment, the tool head 1126 may include a light or an endoscopic camera.

Referring now to FIGS. 11 and 13, the cannulated target arm 1112 may have a distal end 1113 and a proximal end 1115. The cannulated target arm 1112 may have an aperture at both the distal end 1113 and the proximal end 1115 connected by a hollow passageway 1117. The tool head 1126 of the instrument 1120 may be inserted in the aperture in the proximal end 1115 of the target arm 1112 and exit the aperture at the distal end 1113 of the target arm 1112. It will be further appreciated that the distal end 1113 of the target arm 1112 may be inserted into a patient during surgery.

Referring now to FIGS. 13 and 14, where like reference numerals depict like components, the maximum width 1130 of the tool head 1126 may be less than an inner diameter 1132 of the target arm 1112. In an embodiment, the diameter 1132 (FIG. 14) of the cannulation, or the inner diameter of the target arm 1112, may be between 3 millimeters (mm) and 8 mm, or about 4 mm. In an embodiment, an outer diameter 1134 (FIG. 14) of the target arm 1112 may be between 4 mm and 10 mm, or about 5 mm.

Referring now to FIGS. 11, 13 and 14, a foot 1150 may be disposed on a distal end of the target arm 1112. The foot 1150, which may be placed on body tissue, may offset the target arm 1112 from the body tissue, such as a bone or tendon, to prevent tissue damage and to facilitate surgical repair. In an embodiment, the foot 1150 may provide an offset 1136 (FIG. 14) of between 1 mm and 10 mm. It will be appreciated that the cannulated target arm 1112 may eliminate the need for a separate cannulated member that provides access to the surgical site. This may be beneficial because the cannulated target arm 1112 may have a smaller diameter than the separate cannulated member.

Referring now back to FIG. 11, the anchor target area 1105 may be located proximate the distal end 1113 of the target arm 1112. In an embodiment, a guide axis 1208 through the guide 1104 passes proximate the distal end 1113 of the target arm 1112. As used in this paragraph, the term “proximate” may mean within one-half of an inch of the distal end 1113 of the target arm 1112. As used in this paragraph, the term “proximate” may also mean within one inch of the distal end 1113 of the target arm 1112. As can be further observed, the tool head 1126 extends from the distal end 1113 of the target arm 1112. For example, the tool head 1126 of the instrument 1120 may provide a target that extends from the distal end 1113 of the target arm 1112.

In an embodiment, the present disclosure may provide a kit comprising an embodiment of an anchor placement jig according to the present disclosure, one or more suture anchors sized to pass through a barrel of a guide of the anchor placement jig, a suture anchor driver, and optionally one or more instruments with tool heads sized to be inserted through a cannulated target arm, if present, of the anchor placement jig, where all of the foregoing a placed in a sterilized container or package. In use, a surgeon may open the kit during surgery to facilitate repair of a rotator cuff injury.

In the foregoing Detailed Description, various features of the present disclosure are grouped together in a single embodiment for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description of the Disclosure by this reference, with each claim standing on its own as a separate embodiment of the present disclosure.

It is to be understood that the above-described arrangements are only illustrative of the application of the principles of the present disclosure. Numerous modifications and alternative arrangements may be devised by those skilled in the art without departing from the spirit and scope of the present disclosure and the appended claims are intended to cover such modifications and arrangements. Thus, while the present disclosure has been shown in the drawings and described above with particularity and detail, it will be apparent to those of ordinary skill in the art that numerous modifications, including, but not limited to, variations in size, materials, shape, form, function and manner of operation, assembly and use may be made without departing from the principles and concepts set forth herein.

1. An anchor placement jig for partial thickness rotator cuff repair comprising:
a frame;
a target extending from a distal end of the frame, the target comprising:
a perimeter structure;
an aperture extending through the target sized to receive a suture anchor, the perimeter structure extending at least partially around the perimeter;
a perimeter clearance between an exterior border of the perimeter structure of the target, and the aperture, of less than one sixteenth of an inch;
a guide disposed on the frame and aligned with the target, the guide having a guide axis that intersects the aperture of the target.

2. The anchor placement jig of claim 1, wherein the perimeter structure extends less than entirely around the aperture, and wherein the perimeter structure comprises a gap extending into the aperture and running the entire height of the target, the gap sized to allow a suture having two constrained ends to be passed from outside of the perimeter structure to the aperture via the gap.

3. The anchor placement jig of claim 1, wherein the target has a height of less than one sixteenth of an inch.

4. The anchor placement jig or claim 1, wherein the guide is a barrel.

5. The anchor placement jig of claim 1, wherein the aperture has a maximum diameter of between one eighth of an inch and one half of an inch.

6. The anchor placement jig of claim 1, further comprising a guide rotation adjustment mechanism to rotate the guide around the target.

7. The anchor placement jig of claim 1, wherein the target is rotatable relative to the frame.

8. The anchor placement jig of claim 1, further comprising a vertical grip disposed on the frame.

9. The anchor placement jig of claim 1, wherein the frame comprises:
a guide arm extending away from a grip, the guide disposed at a distal end of the guide arm; and
a target arm extending away from the grip, the target disposed at a distal end of the target arm.

10. The anchor placement jig of claim 9, wherein the guide arm and the target arm are separated by a clearance distance of between two and four inches.

11. The anchor placement jig of claim 10, wherein the clearance distance is approximately three inches.

12. The anchor placement jig of claim 9, wherein the guide arm and the target arm are substantially parallel.

13. The anchor placement jig of claim 9, wherein the guide axis is at an angle relative to a long axis of the guide arm of less than ninety degrees.

14. The anchor placement jig of claim 9, wherein a long axis of the guide is at angle relative to a long axis of the guide arm approximately seventy degrees.

15. The anchor placement jig of claim 9, wherein the guide arm has a length of between eight and ten inches.

16. The anchor placement jig of claim 9, wherein the target arm has a length of between seven and nine inches.

17. The anchor placement jig of claim 1, wherein the anchor placement jig comprises stainless steel.

18. The anchor placement jig of claim 1, wherein the frame comprises a cannulation and an elongated portion configured and dimensioned to be removably insertable into the cannulation, therein the target is disposed on a distal end of the elongated portion.

19. An anchor placement jig for partial thickness rotator cuff repair comprising:
a frame;
a target extending from a distal end of the frame, the target comprising:
a perimeter structure;
an aperture extending through the target sized to receive a suture anchor, the perimeter structure extending at least partially around the aperture;
wherein the perimeter structure extends less than entirely around the aperture, and wherein the perimeter structure comprises a gap extending into the aperture and running the entire height of the target, the gap sized to allow a suture having constrained ends to be passed from outside of the perimeter structure to the aperture via the gap;
a guide disposed on the frame and aligned with the target the guide having a guide axis that intersects the aperture of the target.

20. The anchor placement jig of claim 19, wherein the target has a height of less than one sixteenth of an inch.

21. The anchor placement jig of claim 19, wherein the guide is a barrel.

22. The anchor placement jig of claim 19, wherein the aperture has a maximum diameter of between one eighth of an inch and one half of an inch.

23. The anchor placement jig of claim 19, further comprising a guide rotation adjustment mechanism to rotate the guide around the target.

24. The anchor placement jig of claim 19, wherein the target is rotatable relative to the frame.

25. The anchor placement jig of claim 19, further comprising a vertical grip disposed on the frame.

26. The anchor placement jig of claim 19, wherein the frame comprises:
a guide arm extending away from a grip, the guide disposed at a distal end of the guide arm; and
a target arm extending away from the grip, the target disposed at a distal end of the target arm.

27. The anchor placement jig of claim 27, wherein the guide arm and the target arm are separated by a clearance distance of between two and four inches.

28. The anchor placement jig of claim 27, wherein the clearance distance is approximately three inches.

29. The anchor placement jig of claim 26, wherein the guide arm and the target arm are substantially parallel.

30. The anchor placement jig of claim 26, wherein the guide axis is at angle relative to a long axis of the guide arm of less than ninety degrees.

31. The anchor placement jig of claim 26, wherein the guide axis is at angle relative to a long axis of the guide arm approximately seventy degrees.

32. The anchor placement jig of claim 26, wherein the guide arm has a length of between eight and ten inches.

33. The anchor placement jig of claim 26, wherein the target arm has a length of between seven and nine inches.

34. The anchor placement jig of claim 19, wherein the anchor placement jig comprises stainless steel.

35. The anchor placement jig of claim 19, wherein the trains comprises a cannulation and wherein the target is disposed on a distal end of an elongated portion configured and dimensioned to be removably insertable into the cannulation.

36. A system for repairing a partial thickness rotator cuff tear comprising:
an anchor placement jig for partial thickness rotator cuff repair comprising:
a frame;
a target extending from a distal end of the frame, the target comprising:
a perimeter structure;
an aperture extending through the target sized to receive a suture anchor, the perimeter structure extending at least partially around the aperture;
a perimeter clearance between an exterior border of the perimeter structure of the target and the aperture of less than one sixteenth of an inch;
a guide disposed on the frame and aligned with the target, the guide having a guide axis that intersects the aperture of the target;
an anchor attachable to bone, the anchor comprising a suture connection and having a size smaller than the aperture of the target;
a driver removably interfaceable with the anchor, the driver to drive the anchor into bone and sized to fit within the guide.

37. The system of claim 36, wherein the anchor comprises a bone screw, and wherein the driver is configured to rotate the anchor.

38. The system of claim 36, further comprising one or more sutures at the suture connection of the anchor.

39. A method for repairing a partial-thickness rotator cuff tear comprising the steps of:
abrading the humerus in an abrasion region near a partially attached rotator cuff tendon;
providing an anchor placement jig for partial thickness rotator cuff repair comprising:
a frame;
a target extending from a distal end of the frame, the target comprising:
a perimeter structure;
an aperture extending through the target sized to receive a suture anchor, the perimeter structure extending at least partially around the aperture;
a perimeter clearance between an exterior border of the perimeter structure of the target and the aperture of less one sixteenth of an inch;
a guide disposed on the frame and aligned with the target, the guide having a guide axis that intersects the aperture of the target;
positioning the target at a desired locations on the abrasion region;
inserting an anchor through the guide and the aperture;
attaching the anchor to the humerus in toe abrasion region;
securing a tendon to the anchor with one or more sutures.

40. The method of claim 39, further comprising inserting the target through an anterior portal of a patient.

41. The method of claim 39, further comprising retrieving the sutures attached to the anchor using the target.

42. The method of claim 39, wherein attaching the anchor to the humerus comprises rotating the anchor.

43. The method of claim 39, further comprising inserting a needle through a rotator cuff tendon of a patient to a position near the abrasion region.

44. The method of claim 43, wherein the needle is an eighteen gauge needle.

45. The method of claim 43, further comprising passing a loop through the needle to a position near the abrasion region and retrieving a suture with the loop through the needle.

46. The method or claim 39, further comprising tightening a suture connected to the anchor to draw the tendon to the abrasion region.

47. The method of claim 39, further comprising: tying the suture to secure the tendon at the abrasion region.

48. The method of claim 39, wherein the frame comprises a cannulation and wherein the target is disposed on a distal end of an elongated portion configured and dimensioned to be removably insertable into the cannulation.

49-64. (canceled)
